(19) United States
(12) Patent Application Publication
(10) Pub. No.: US 2008/0025972 A1
(43) Pub. Date: Jan. 31, 2008

(54) Treating sex steriod responsive disorders

(75) Inventors: Yehia Daaka, Chapel Hill, NC (US); Jonathan S. Stamler, Chapel Hill, NC (US)

Correspondence Address: BACON & THOMAS, PLLC, 625 SLATERS LANE, FOURTH FLOOR, ALEXANDRIA, VA, 22314, US

(73) Assignee: Duke University, Durham, NC (US)

(21) Appl. No.: 11/802,116

(22) Filed: May 21, 2007

Related U.S. Application Data
(60) Provisional application No. 60/833,274, filed on Jul. 26, 2006.

(51) Int. Cl. A61K 31/34; A61K 31/04; A61K 31/135; A61K 31/16; A61K 31/445; A61K 31/7052; A61K 38/06; A61P 35/00; G01N 33/53; A61K 39/395; A61K 38/02; A61K 31/497; A61K 31/415; A61K 31/195; A61K 31/335

(52) U.S. Cl. 424/130.1; 435/7.1; 514/18; 514/2; 514/252.2; 514/328; 514/391; 514/397; 514/44; 514/449; 514/470; 514/562; 514/630; 514/656; 514/740; 514/742

(57) ABSTRACT

Sex steroid potentiated disorders including, prostate cancer and breast cancer, in a patient in need of treatment thereof, are treated with an amount of nitric oxide donating compound and/or nitrosoglutathione reductase inhibitor and/or cysteine binder different from that provided by nitric oxide donating compound effective to inhibit activation of steroid receptor. Variations include using only nitric oxide donating agent as treating agent; using only nitrosoglutathione reductase inhibitors as treating agent, using nitric oxide donating agent plus nitrosoglutathione reductase inhibiting agent; for prostate cancer treatment using prostate cancer drug modified to contain nitric oxide donating moiety or FDA approved nitric oxide donating agent and FDA approved prostate cancer treating agent. Also disclosed is an assay for assessing mutagenic potential of prostate cancer in a patient.

### CROSS-REFERENCE TO RELATED APPLICATIONS

This application claims the benefit of U.S. Provisional Patent Application 60/833,274, filed Jul. 26, 2006.

### TECHNICAL FIELD

This invention is directed to treating sex steroid potentiated disorders, e.g. prostate cancer or breast cancer, and to an assay for assessing the mutagenic potential of prostate cancer.

### BACKGROUND OF THE INVENTION

It is well established that androgen receptor stimulation promotes prostate cancer and that estrogen receptor stimulation promotes breast cancer. The targeting of steroid receptors is standard treatment for these cancers. However, current modes of therapy are only partly effective.

Moreover, it is known that some prostate cancers are non-aggressive. The reasons for this are not known. It is posited herein that a reason for this is that in non-aggressive prostate cancer tumors, the androgen receptor is quelled.

### SUMMARY OF THE INVENTION

It has been discovered herein that nitric oxide and nitric oxide related compounds, that is compounds able to transfer NO′, NO⁺, NO⁻ or NO₂⁺ group to biological molecules, inhibit sex steroid receptor activation, e.g., androgen and estrogen receptor activation, and thereby suppress the progression of sex steroid potentiated disorders, e.g., prostate cancer and breast cancer, and that this is a reason why some prostate cancer tumors are not aggressive, i.e., the non-aggressive tumors vigorously express or over-express eNOS and/or iNOS.

In one embodiment herein, denoted the first embodiment, the invention is directed to a method of treating a sex steroid potentiated disorder in a patient in need of such treatment. The method comprises administering to said patient an amount of treating agent comprising nitric oxide (NO) donating compound effective to inhibit activation of receptor otherwise activated by the steroid. The nitric oxide donating compound is not a substrate for glutathione-S-transferase. In one case, for example, the disorder is prostate cancer and administration of nitric oxide donating compound is effective to inhibit activation of androgen receptor (AR). In another case, for example, the disorder is breast cancer and administration of the nitric oxide donating compound is effective to inhibit activation of estrogen receptor.

In another embodiment herein, denoted the second embodiment, the disorder of the first embodiment is prostate cancer and the NO donating compound of the first embodiment is obtained by providing NO donating moiety on a prostate cancer treating agent which does not otherwise have NO donating effect.

In another embodiment herein, denoted the third embodiment, the disorder of the first embodiment is prostate cancer and the treating agent of the first embodiment comprises nitric oxide donating compound and also a nitrosoglutathione reductase inhibitor.

In another embodiment herein, denoted the fourth embodiment, the disorder of the first embodiment is prostate cancer and the treating agent of the first embodiment comprises an amount of a nitric oxide donating compound which is an FDA approved or foreign corresponding agency approved nitrate and an amount of an FDA approved or foreign corresponding agency approved prostate cancer treating agent, effective to inhibit prostate cancer cell proliferation and/or provide palliative effect for the prostate cancer.

In another embodiment herein denoted the fifth embodiment, the invention is directed to a method of treating a sex steroid potentiated disorder in a patient in need of such treatment, comprising administering to the patient an amount of glutathione reductase inhibitor effective to inhibit activation of receptor for the steroid.

In another embodiment herein, denoted the sixth embodiment, the invention is directed to a method of treating a sex steroid potentiated disorder in a patient in need of such treatment, comprising administering amount of NO donating compound and administering amount of treating agent comprising compound that provides moiety that binds to cysteine of the receptor of the steroid which is not NO donating compound, effective to inhibit activation of the receptor for the steroid.

Yet another embodiment herein, denoted the seventh embodiment, is directed to a method of assessing mutagenic potential of prostate cancer in a patient comprising the steps of
(a) obtaining prostate cancer tissues/cells from the patient,
(b) incubating the cancer tissues/cells obtained in step (a) or cells cultured therefrom with anti-nitrosylated androgen receptor antibody or anti S-nitrosocysteine antibody,
(c) determining whether antigen antibody reaction occurs,
(d) where the occurrence of said reaction indicates low mutagenic potential.

As used herein, the term “steroid potentiated” means that endogenous steroid or the administration of steroid promotes pathological proliferation of cells.

The term “sex steroid” is used herein to be synomous with the term “gonadal steroid”. The sex steroids are steroid hormones which interact with vertebrate androgen or estrogen receptors. Natural sex steroids are made by the gonads (ovaries or testes), by adrenal glands, or by conversion from other sex steroids in other tissues such as liver or fat. The two main classes of sex steroids are androgens and estrogens, of which the most important human examples are testosterone and estradiol, respectively. A third class of sex steroids distinct from androgens and estrogens is progestagen (progesterone is the most important and only naturally occurring human progestagen). There are many synthetic sex steroids. Synthetic androgens are often referred to as anabolic steroids. Synthetic estrogens and progestagens are used in oral contraception pills. Diethylstilbestrol (DES) is a synthetic estrogen. Sex steroids include, for example, androgens: testosterone, androstenedione, dihydrotestosterone, dehydroepiandrosterone, anabolic steroid; estrogens: estradiol, diethylstilbestrol; progestagens: progesterone; progestins.

The terms “nitric oxide donating compound” and “NO donating compound” are used herein to means compound that has the ability to transfer NO′, NO⁺, NO⁻ or NO₂⁺ to cysteine of a steroid receptor.

The terms “providing nitric oxide donating moiety” and “providing NO donating moiety” are used herein to refer to providing a moiety on a prostate cancer treating agent, that has the ability to transfer NO′, NO⁺, NO⁻ or NO₂⁺ to cysteine of a steroid receptor.

The terms “nitric oxide donating effect” and NO donating effect” are used herein to mean effect of causing transfer of NO′, NO⁺, NO⁻ or NO₂⁺ to cysteine of a steroid receptor.

The term “activation of a sex steroid receptor” is used herein to mean impart ability to the steroid receptor to bind to the corresponding steroid and effect transcriptional output of said receptor or mediate growth of sex steroid stimulated cancer cells.

Amount effective to inhibit activation of a sex steroid receptor can be determined by treatment of animal or human with androgen receptor antagonist (or estrogen receptor antagonist or other relevant sex steroid receptor antagonist).

Nitrosoglutathione reductase inhibition and nitrosoglutathione reductase inhibition function are determined by direct measurement of enzyme activity as described in Liu, et al., “Essential roles of S-nitrosothiols in vascular homeostasis and endotoxic shock”, Cell 116, 617-628 (2004) or as described in Thoenges, D., et al., Journal of Biomolecular Screening 7 (4), 353-357 (2002).

The term “mutagenic potential” is used herein to mean potential of cancer cells to proliferate and/or to mediate metastasis.

### DETAILED DESCRIPTION

We turn now to the first embodiment of the invention. Examples of sex steroid potentiated disorders include prostate cancer, breast cancer and uterine cancer.

The NO donating compound can be, for example, a nitrate or a nitrite. For example, the NO donating compound can be selected from the group consisting of isosorbide mononitrate, isosorbide dinitrate, ethyl nitrite, amyl nitrite, nitroglycerin, a nitrosothiol, and nitroprusside and combinations thereof. Examples of nitrosothiols are nitrosoglutathione and S-nitroso acetylpenicillamine.

We turn now to the dosage of NO donating compound where the NO donating moiety is not associated with an otherwise therapeutic agent. As indicated above, the dosage is amount effective to inhibit activation of the receptor for the sex steroid potentiating the disorder treated and this is determined ex vivo by determining concentration of NO donating compound that inhibits activation by sex steroid of sex steroid receptor to inhibit proliferation of pathologically proliferating cells by at least 50% and providing this concentration or at least 10% of this concentration in blood. In the case where the sex steroid potentiating disorder is prostate cancer, the sex steroid is androgen and the sex steroid receptor is androgen receptor and the dosage of NO donating compound is amount effective to inhibit activation of androgen receptor. In the case where the sex steroid potentiated disorder is breast cancer, the dosage of NO donating compound is amount effective to inhibit activation of estrogen receptor. Where the NO donating compound is FDA approved, the FDA approved dosage is used if this meets the above determination of inhibition of steroid receptor activation result. For isosorbide mononitrate a suitable dosage ranges from, for example, 5 mg to 250 mg. For isosorbide dinitrate a suitable dosage ranges, for example, from 5 mg to 250 mg.

Route of administration for the nitrates and nitrites can be, for example, sublingual, topical, intravenous or oral. For isosorbide mononitrate a preferred route of administration is oral. For isosorbide dinitrate a preferred route of administration is oral.

We turn now to the second embodiment herein which is directed to a method of treating prostate cancer in a patient in need of such treatment, comprising administering to said patient an amount of NO donating compound effective to inhibit androgen receptor activation where the NO donating compound is obtained by providing NO donating moiety on a prostate cancer treating agent which does not otherwise have NO donating effect. The prostate cancer drugs that do not otherwise have NO donating effect include, for example, luteinizing hormone-releasing hormone (LHRH) agonists, antiandrogens, adrenal blockers, estrogens, docetaxel plus prednisone, and mitoxantrone plus prednisone. Luteinizing hormone-releasing hormone agonists which do not otherwise have NO donating effect, include for example, leuprolide, goserelin, buserelin, and abaralix. Antiandrogens which do not otherwise have NO donating effect include, for example, bicalutamide and nilutamide and flutamide (there is no evidence that flutamide is an NO donor). Adrenal blockers which do not otherwise have NO donating effect include, for example, ketoconazole and aminoglutethimide. Estrogens which do not otherwise have NO donating effect are diethylstilbestrol (DES) and ethynyl estradiol. All the drugs specifically listed in this paragraph as not otherwise having NO donor effect are FDA approved for treatment of prostate cancer.

NO donating moiety is readily provided on the listed drugs by substitution of a primary or secondary amino group with a hydroxy acid (e.g., glycolic acid followed by conversion of the hydroxyl groups to nitrite esters or nitrate esters; by conversion of a secondary amino function to NONOate species which release NO some fraction of which is converted to nitrosylating agent in vivo; by substitution of primary and secondary amino groups or hydroxyl groups with a thiol acid (e.g., thiolacetic acid) to provide the corresponding amide or ester and provide a thiol group which is converted to S-nitrosothiol by treatment with acidified sodium nitrite or alkyl nitrite; by converting ketone to corresponding enamine, then amidating with carboxylic acid bearing a secondary amine and proceeding as for a secondary amine as described above; by converting hydroxyl group with NO to nitrite ester or with NO₂ to nitrate ester; or by converting thiol group to S-nitrosothiol by reaction with acidified sodium nitrite or alkyl nitrite.

Bicalutamide is a preferred prostate cancer drug to add NO donating moiety to, e.g., —ONO or —ONO₂.

The prostate cancer drugs that are provided with NO donating moiety thereon not only have the therapeutic effect of the starting drug but also provide NO donating effect, i.e., are NO donating compounds.

The dosage for the NO donating compound prostate cancer drug of the second embodiment is preferably the same as for or up to 90 percent less than for the same prostate cancer drug without NO donating moiety thereon and the route of administration for NO donating compound prostate cancer drug of the second embodiment is the same as for the same prostate cancer drug without NO donating moiety thereon.

We turn now to the third embodiment herein.

The NO donating compound for the third embodiment and the dosage and route of administration for it can be the same as for the first and second embodiments herein.

We turn now to the nitrosoglutathione reductase inhibitors for the third embodiment. Nitrosoglutathione reductase inhibition function can be determined as indicated above. Nitrosoglutathione reductase inhibitors for the third embodiment include, for example, the inhibitors of glutathione-dependent formaldehyde dehydrogenase described in U.S. Publication no. 2002-0128205 A1, and WO 2002/055018 A2, including D-glutathione, ribavirin, D-glutathione together with ribavirin and mycophenolic acid and combinations thereof using the dosages and routes of administration stated therein. The whole U.S. Publication No. 2002-0128205 A1 and WO 2002/055018 A2 are incorporated herein by reference.

We turn now to the fourth embodiment herein.

FDA approved nitric oxide donating compounds as well as dosages thereof and routes of administration for these are named in the description of the first embodiment. FDA and foreign corresponding agency approved nitric oxide donating compounds include, for example, sodium nitroprusside, nitroglycerin, isosorbide nitrates and nicorandil.

FDA approved prostate cancer treating drugs include, for example, the luteinizing hormone-releasing hormone agonists leuprolide, goserelin, buserelin, abaralix; the antiandrogens flutamide, bicalutamide, nilutamide; the adrenal blockers ketoconazole and aminoglutethimide; the estrogens diethylstilbestrol and ethynyl estradiol; docetaxel plus prednisone; mitoxantrone plus prednisone. The prostate cancer drugs can be administered in the dosages and with the routes of administration associated with FDA approval of them.

We turn now to the fifth embodiment. Nitrosoglutathione reductase inhibition function can be determined as indicated above. Nitrosoglutathione reductase inhibitors for this embodiment and the dosages and routes of administration of them include those indicated above for the third embodiment.

We turn now to the sixth embodiment. The NO donating compounds are those described for the first embodiment. The cysteine binding treating agents include, for example, diethylmaleate, sodium tetrathionate, thioredoxin, vincristine, vinblastine, colchicine, paclitaxel, and RSO₂—SO(═)NEt₂ where R is ortho-O₂N—C₆H₄— or para-O₂N—C₆H₄—.

Dosages and routes of administration for NO donating compounds are those described for the first embodiment. Dosages and routes of administration for the cysteine binding agents are those approved by the FDA for any purpose or if not yet FDA approved, the dosages and routes of administration used for testing for FDA approval.

We turn now to the seventh embodiment.

Prostate cancer cells are obtained by biopsy from the patient followed by tissue preparation (both frozen and fixed) for sectioning.

Anti-nitrosylated androgen receptor antibody is obtained by purchasing androgen receptor, nitrosylating it by treatment with S-nitrosocysteine and following standard approaches to raising antibody.

Anti-S-nitrosocysteine antibody is obtainable commercially.

The prostate cancer cells or cultured cells therefrom are incubated with S-nitrosothiol (SNO) antibody diluted in phosphate buffered saline (PBS) solution at 4° C. for overnight.

The occurrence of antigen antibody reaction is determined by ELISA.

The invention is supported by the following Background Examples and illustrated by the following Working Examples.

### BACKGROUND EXAMPLE 1

### Proving Colocalization of e-NOS and Androgen Receptor in Prostate Cells

LNCaP prostate cells obtained from American Type Culture Collection fixed by incubation in, e.g., 10%, formalin solution were incubated with antibody (monoclonal anti-AR antibody obtained from Sanzt Crus Biotech) that recognizes androgen receptor. The procedure is as follows.

After fixation with formalin and then two washes with phosphate buffered saline (PBS), cells are incubated for 5 minutes with 0.5% Triton X100. After two washes with PBS, the cells are incubated overnight at 4° C. with AR antibody (SC-7305 Santa Cruz Biotech; 1:50 dilution). Next the cells are incubated with secondary antibody, FITC (fluorescein isothiocyanate form) conjugated donkey anti mouse IgG;1:200 dilution (Jackson ImmunoResearch 715-096-151), for 45 minutes at room temperature. After one wash with PBS, cells are incubated with Hoechst for nuclear staining, before mounting and visualization under microscope. Recognition of androgen receptor is indicated by green color conjugating the cells.

Fixed prostate cells as described above were incubated with antibody (rabbit polyclonal antibody, Santa Cruz Biotech, SC-654), that recognizes e-NOS, linked to a red color. Presence of e-NOS is indicated by red color conjugating to cells.

Colocalization (the presence of two or more molecules in the same location in a specimen), in this case androgen receptor and e-NOS, in prostate cells is indicated by yellow color when the cells are incubated with both the antibodies.

### BACKGROUND EXAMPLE 2

### Showing that e-NOS is Bound to Androgen Receptor in Prostate Cells

Antibody (rabbit polyclonal antibody, Santa Cruz Biotech, SC-654), was added to prostate cells (LNCaP cells from ATCC) to form complex with e-NOS and all proteins binding it in said cells. The antibody-protein complex was pelleted using protein-G/plus protein-A sepharose. Immunoblotting with antibody (anti-AR antibody; Santa Cruz Biotech) that recognizes androgen receptor showed androgen receptor bound to e-NOS. Protein bands were visualized using enhanced chemiluminescence (ECL).

### BACKGROUND EXAMPLE 3

### Showing that Androgen Receptor Can Be S-Nitrosylated

Purified androgen receptor protein (obtained from Cambridge Bioscience, England) was exposed to nitric oxide by means of contact with nitrosoglutathione (GSNO) under anaerobic conditions. Glutathione (GSH) was used as a control. S-Nitrosylation of androgen receptor was detected by the biotin switch assay as described by Jeffrey, S. R., et al., Nat. Cell Biol 3, 193-197 (2001).

### BACKGROUND EXAMPLE 4

### Showing that S-Nitrosylation of Androgen Receptor Affects Androgen Receptor Function

A luciferase reporter assay as described in Kasbohm, E. A., et al., Journal of Biological Chemistry 289,11583-11589 (2005), was used to detect androgen receptor activation.

As a control, the following was carried out. Androgen receptor in the form of prostate cancer cells was incubated with androgen under the following conditions.

Cells were seeded in 100-mm dishes and transfected at 70% confluence using DMRIE-C Reagent (Invitrogen). Transfections were carried out with cDNAs encoding the probasin or ARR2 luciferase (1 μg) and SV40-Renilla luciferase reporter plasmid (Promega) (10 ng) together with empty vector or the indicated gene of interest using a total of 6 μg of DNA/plate. At the end of transfection, cells were equally divided into 6-well plates and allowed to attach. The culture medium was replaced after 24 hours with starvation medium (phenol red-free RPMI 1640 cell culture medium) containing 5% charcoal-stripped fetal bovine serum, 1% penicillin/streptomycin, and 10 mM HEPES buffer, pH 7.5), and identical cell populations, in duplicate, were stimulated with androgen or vehicle for an additional 24 hours. Luciferase activities in cell lysates were measured using the Dual Luciferase assay system (Promega; Madison, Wis.) and were normalized by the Renilla activities and protein concentrations of the samples. Luciferase reporter assay showed that androgen activated androgen receptor.

In a second case, L-NAME was included in the incubation mixture to inhibit nitric oxide production. Luciferase reporter assay showed that androgen receptor was activated by androgen at concentrations that do not otherwise activate androgen receptor. This shows that nitric oxide synthesis inhibition dramatically potentiated androgen receptor activation. This indicates that patients on endocrine blocking therapy (who will still have a low concentration of androgen) will possess prostate cancer cells with activated androgen receptor and suggests why androgen synthesis blockers are not entirely effective in ameliorating prostate cancer.

In a third case, prostate cancer cells were transfected with exogenous e-NOS by using human eNOS cDNA. Luciferase reporter assay showed that even at high androgen concentrations, there was no maximal activation of androgen receptor.

The above suggests that nitric oxide impairs androgen receptor activation.

### BACKGROUND EXAMPLE 5

### Showing That S-Nitrosylation of Androgen Receptor Causes Reduction in Prostate-Specific Antigen (PSA) Expression

The experiments of Background Example 4 were repeated but instead of determining androgen receptor activation, PSA level was measured by PSA protein expression in LNCaP cells. LNCaP cells were incubated in phenol red-free RPMI cell culture medium containing 5% charcoal-stripped fetal bovine serum (CSS) for 48 hours and then treated with dihydrotestosterone (DHT) (10 nmol/L) or vehicle control (ethanol) for 24 hours. Immunoblots were conducted using anti-PSA antibodies to measure PSA protein expression. Same filters were immunoblotted using anti-actin antibodies as control to establish equal loading of proteins.

The experiments showed that when nitric oxide synthesis was inhibited (L-NAME case), more PSA was expressed. These experiments also showed that when eNOS was overexpressed, cells expressed less of the PSA protein.

### BACKGROUND EXAMPLE 6

### Comparison of Prostate Cancer Cell Growth Using Wild Type and e-NOS Overexpressing Prostate Cancer Cells

Cell proliferation was determined in cellular assays carried out as follows:

LNCaP cells (obtained from ATCC) and seeded at 2.5×10⁴/well were incubated in charcoal-stripped serum for 24-48 hours prior to stimulation with DHT (5-10 nmole/L) for an additional 48 hours. The number of viable cells (that exclude trypan blue) was counted using a light microscope and a hemocytometer.

With wild type prostate cancer cells (obtained from ATCC), androgen at a concentration of 5-10 nmol/L caused increased cell growth.

With e-NOS overexpressing prostate cancer cells (obtained as in Background Example 4) androgen at concentrations of 5-10 nmol/L had no effect on cell growth.

### BACKGROUND EXAMPLE 7

### In Vivo Tumor Growth

Three kinds of prostate cancer cells were implanted in mice. These were (1) parental wild type LNCaP cells (obtained from ATCC), (2) e-NOS overexpressing prostate cancer cells (obtained as in Background Example 4), i.e., LNCaP cells that stably overexpress human eNOS gene, and (3) LNCaP cells that have knocked down expression of human eNOS gene (no e-NOS function). The cells (3) were obtained using siRNA (small interfering RNA, a double stranded RNA composition designed to act primarily through RNA interference). The siRNA was obtained by synthesizing complementary RNA sequences that specifically recognize that human eNOS gene and was used to knock down e-NOS gene by stably expressing the RNA interference (RNAi) in LNCaP cells.

Each of the three kinds of prostate cancer cells were implanted in groups of mice by xenograft implanation as described by Bookout, A. L., et al., Journal of Biological Chemistry 278, 37569-37575 (2003).

Tumor growth in each group was measured by calipers and measurements were converted to tumor volume using the formula V=π/6×S×S×L, where S is the short dimension of the tumor, and L is the long dimension of the tumor.

Tumor growth was fastest in the e-NOS silenced group (androgen receptor was always active since there was no nitric oxide to interfere with androgen receptor activation).

There was no tumor growth in the e-NOS overexpressing implanted group.

### WORKING EXAMPLE I

A 60-year-old male with prostate cancer and an elevated PSA level is treated with isosorbide mononitrate, 40 mg PO BID, for 3 months and the PSA level declines by 50%.

### WORKING EXAMPLE II

A 65-year-old male with prostate cancer and an elevated PSA level is treated with isosorbide dinitrate, 20 mg PO BID, and the PSA level stabilizes over 3 months.

### WORKING EXAMPLE III

A 63-year-old male with prostate cancer and body metastases is treated with busaralin but the disease progresses. Topical nitroglycerin, 0.8 mg/hour, is administred and progression of the disease is measured by PSA and bony metastases slows.

### WORKING EXAMPLE IV

A 70-year-old male with an elevated PSA is treated with oral S-nitrosoglutathione (40mg PO BID) and the PSA level decreases over 3 months by 50%.

### WORKING EXAMPLE V

A 50-year-old with prostate cancer is treated with flutamide provided with an NO donating moiety, 250 mg orally 3 times/day, and PSA level drops by 50% over a month.

### WORKING EXAMPLE VI

A 60-year-old with prostate cancer is treated with prednisone provided with an NO donating moiety, 5 mg 4 times/day, and the patient's bone pain and fatigue improve over a month.

### WORKING EXAMPLE VII

A 60-year-old white male is treated with bicalutamide provided with an NO donating moiety, 150 mg/day and the patient responded by an improvement in performance status, bone pain, and decreased need for analgesia during his therapy.

### WORKING EXAMPLE VIII

A 90-year-old with prostate cancer is treated with ketaconazole provided with an NO donating moiety, 400 mg 3 times/day, and the patient's PSA level drops by 60% over 4 months.

### WORKING EXAMPLE IX

A 75-year-old white male is treated with a combination of ketaconazole, 200 milligrams 3 times/day and isosorbide dinitrate 40 mg twic/day, and PSA level drops by 50% over 3 months.

### WORKING EXAMPLE X

A 27-year-old white female with breast cancer is treated with isosorbide mononitrate, 40 mg PO BID, and lung metastases regress over 6 months.

### WORKING EXAMPLE XI

A 50-year-old white male with prostate cancer is given D-glutathione, 150 mg orally 4 times/day, and the PSA level drops by 50% over 4 months.

### WORKING EXAMPLE XII

To assess mutagenic potential, a biopsy obtained of the prostate is stained with an antinitrosocysteine antibody which shows no staining. The patient is started on isosorbide mononitrite, 40 mg PO BID.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

What is claimed is:

1. A method of treating a sex steroid potentiated disorder in a patient in need of such treatment, comprising administering to said patient an amount of treating agent comprising nitric oxide donating compound effective to inhibit activation of receptor otherwise activated by the steroid, provided the nitric oxide donating compound is not a substrate for glutathione-S-transferase.

2. The method of claim 1 where the disorder is prostate cancer and administration of the nitric oxide donating compound is effective to inhibit activation of androgen receptor.

3. The method of claim 2 where the NO donating compound is a nitrate or a nitrite.

4. The method of claim 3 where the NO donating compound is selected from the group consisting of isosorbide mononitrate, isosorbide dinitrate, ethylnitrite, amylnitrite, nitroglycerin, a nitrosothiol and nitroprusside and combinations thereof.

5. The method of claim 3 where the NO donating compound comprises nitrosoglutathione.

6. The method of claim 3 where the NO donating compound is obtained by providing NO donating moiety on a prostate cancer treating agent which does not otherwise have NO donating effect.

7. The method of claim 6 where the prostate cancer treating agent which does not otherwise have NO donating effect, is a luteinizing hormone-releasing hormone agonist.

8. The method of claim 6 where the prostate cancer treating agent which does not otherwise have NO donating effect, is an anti-androgen receptor antibody.

9. The method of claim 6 where the prostate cancer treating agent which does not otherwise have NO donating effect, is an adrenal blocker.

10. The method of claim 1 where the treating agent also comprises a nitrosoglutathione reductase inhibitor.

11. The method of claim 10 where the nitrosoglutathione reductase inhibitor is selected from the group consisting of D-glutathione, ribavirin, mycophenolic acid and combinations thereof.

12. The method of claim 2 where said treating agent comprises an amount of a nitric oxide donating compound which is an FDA approved nitrate and an amount of FDA approved prostate cancer treating agent, effective to inhibit prostate cancer cell proliferation and/or provide palliative effect for the prostate cancer.

13. The method of claim 12 where the FDA approved prostate cancer treating agent is selected from the group consisting of leuprolide, goserelin, buserelin, abaralix, flutamide, bicalutamide, nilutamide, ketoconazole, aminoglutethimide, diethylstilbestrol, ethinyl estradiol, docetaxel plus prednisone and mitoxantrone plus prednisone.

14. The method of claim 13 where the FDA approved nitrate is isosorbide mononitrate in a dosage ranging from 5 mg to 250 mg or isosorbide dinitrate in a dosage ranging from 5 mg to 250 mg.

15. The method of claim 1 where the disorder is breast cancer and administration of the nitric oxide donating compound is effective to inhibit activation of estrogen receptor.

16. A method of treating a sex steroid potentiated disorder in a patient in need of such treatment, comprising administering to the patient an amount of glutathione reductase inhibitor effective to inhibit activation of receptor otherwise activated by the steroid.

17. The method of claim 16 where the disorder is prostate cancer.

18. A method of treating a sex steroid potentiated disorder in a patient in need of such treatment, comprising administering amount of NO donating compound and amount of treating agent comprising compound that provides moiety that binds to cysteine of the receptor of the steroid which is not an NO donating compound, effective to inhibit activation of receptor of the steroid.

19. The method for assessing mutagenic potential of prostate cancer in a patient comprising the steps of
(a) obtaining prostate cancer tissues/cells from the patient,
(b) incubating the cancer tissues/cells obtained in step (a) or cells cultured therefrom with anti-nitrosylated androgen receptor antibody or anti-S-nitrosocysteine antibody,
(c) determining whether antigen antibody reaction occurs, and
(d) where the occurrence of said reaction indicating low mutagenic potential.
